(19) United States
(12) Patent Application Publication
(10) Pub. No.: US 2017/0325520 A1
(43) Pub. Date: Nov. 16, 2017

(54) THERMOCHROMIC APPAREL FOR DETECTING PHYSICAL EXHAUSTION AND PROCESS FOR PREPARATION THEREOF

(71) Applicant: CORNELL UNIVERSITY, Ithaca, NY (US)

(72) Inventors: Chih-Chang CHU, Ithaca, NY (US); Alicia POTUCK, Towanda, PA (US); Sarah MEYERS, Warren, NJ (US); Ariana LEVITT, Brooklyn, NY (US); Eric BEAUDETTE, Hollis, NH (US); Xiao HONG, Beijing (CN)

(73) Assignee: CORNELL UNIVERSITY, Ithaca, NY (US)

(21) Appl. No.: 15/529,971

(22) PCT Filed: Nov. 25, 2015
(86) PCT No.: PCT/US15/62747

§ 371 Date: May 25, 2017

Related U.S. Application Data
(60) Provisional application No. 62/084,462, filed on Nov. 25, 2014.

(51) Int. Cl. A41D 13/00; A61B 5/01; A41D 31/00; A61B 5/00

(52) U.S. Cl. CPC A41D 13/0015; A61B 5/6814; A61B 5/6828; A41D 31/0011; A61B 5/015; A61B 2503/10

(57) ABSTRACT

The present invention relates to a thermochromic garment comprising a thermochromic pigment. The present invention also relates to a process for making a thermochromic garment comprising a thermochromic pigment. The present invention further provides methods for using a thermochromic garment comprising a thermochromic pigment, including, inter alia, methods involving monitoring changes in temperature of a human subject or a portion of the human subject that is in contact with the garment. The present invention also provides an apparatus for measuring temperature changes at or near one or more areas of a human subject, where the apparatus comprises a thermochromic pigment based garment.

### CROSS-REFERENCE TO RELATED APPLICATIONS

This application claims priority benefit of U.S. Provisional Patent Application Ser. No. 62/084,462, filed Nov. 25, 2014, the disclosure of which is hereby incorporated by reference herein in its entirety.

### FIELD OF THE INVENTION

The present invention relates to a thermochromic garment comprising a thermochromic pigment, a process for making a thermochromic garment comprising a thermochromic pigment, and methods for using a thermochromic garment comprising a thermochromic pigment. The present invention also provides thermochromic apparel for detecting physical exhaustion and processes for preparing same.

### BACKGROUND OF THE INVENTION

Since the 1990s, various research and development efforts have been made in an attempt to integrate technology into fashion, which has expanded the meaning of clothing from the icon of aesthetics or daily necessity to a wearable form of human performance enhancement. One category of popular and functional garments is performance athletic wear, specifically geared toward athletic applications and sports enthusiasts. The biggest challenge in sportswear design is providing thermal comfort while the human body experiences elevated body temperatures and perspiration during athletic activity. Paralleling functionality, marketing analysis of the athletic apparel industry found comfort, fit, and style are the most desirable traits for athletic apparel, with a higher percentage of users preferring garments that can translate through both sports and casual wear (Fowler 1999).

Athletic wear is designed to anticipate the needs of its wearer including flexibility, durability, and breathability (Mukhopadhyay 2008). The combination of technology and apparel design can further optimize wearer experience and meet the rigorous demands of athletes. For example, rise of heart rate and body temperature in marathon athletes is of great concern during arduous energetic output. To monitor real time physiological parameters, researchers (Mitsubayashi 2003, Morris 2009, Windmiller 2013) described novel ways to merge “smart apparel” with medical functionalities, including the “Smart shirt” which contains a motherboard within the apparel garment capable of monitoring and recording patient heart rate, respiration rate, and pulse oximetry for applications ranging from military, civilian, space, and safety. Although comprehensive physiological data can be collected, these systems suffer from pitfalls of ungainly electronic connections, cost, maintenance, and durability, making these systems not widely accessible.

Monitoring physical exhaustion of the wearer is pivotal in aiding healthy non-destructive exercise. Previous studies show fatigued athletes exhibited reduced cardiac output, central blood volume, and stroke volume, characteristic of the detrimental influence attributed to physical exhaustion (Gonzalez-Alonso 1997, Gonzalez-Alonso 1999). Similarly, heart rate at the apex of fatigue is approximately 98-99% of maximal heart rate. Mean temperatures taken of skin during fatigue (avg. of measurements taken of upper arm, forearm, chest, back, thigh, and calf) correlate increased mean skin temperature (37.2±0.01° C.) and physical exhaustion (Gonzalez-Alonso 1999). Research indicates resting abdominal skin temperatures of normal weight subjects to be 32.8±0.03° C. (Savastano 2009).

Looking more closely at this colorimetric technology, thermochromic pigments are leuco dyes that have become popular due to their color changing abilities (Kul\check{c}ar 2010, Zhu 2005). When the temperature surrounding the pigments rises, the solvent present within the pigment microcapsules undergoes transition to liquid phase, followed by salt dissociation, pH shift, and protonation of the leuco dye resulting in a significant shift in the absorption of the molecule, leading to a colorless appearance. Depending on the temperature of the transition, the pigments can appear colored or colorless on the textile material. In order to apply this technology to apparel design and fabrication, thermochromic pigments can be applied onto the surface of the textile material (Mather 2001, Tang 2006, White 1999), then used to create an overall athletic apparel piece. Unlike smart clothing that has already been developed, thermochromic pigments offer a new design solution for functional sportswear that does not require external electronic components and color change is based solely on the physiological indications (skin temperature) given by the wearer.

The present invention is directed to overcoming these and other deficiencies in the art.

### SUMMARY OF THE INVENTION

The present invention generally relates to, inter alia, thermochromic pigment based apparel, processes for making such apparel, and methods of using such apparel.

According to one aspect, the present disclosure provides a thermochromic garment comprising a thermochromic pigment. In one embodiment, the thermochromic pigment of the thermochromic garment comprises any pigment that turns color in response to a change in temperature. More particularly, the thermochromic pigment of the thermochromic garment can include, without limitation, a leuco dye.

According to another aspect, the present disclosure provides a process for making a thermochromic garment comprising a thermochromic pigment. In one embodiment, the process comprises applying a thermochromic pigment to at least one portion of a non-pigment-treated garment.

According to another aspect, the present disclosure provides a method for using a thermochromic garment comprising a thermochromic pigment. In one embodiment, the method involves monitoring changes in temperature of a human subject or a portion of the human subject that is in contact with the thermochromic garment. In particular embodiments, the method relates to monitoring or treating various conditions or aspects of the human subject selected from the group consisting of physical exhaustion, diabetes, arthritis, general ulcers (e.g., skin ulcers), pressure ulcers, and changes in temperature of portions of the human subject.

According to another aspect, the present disclosure provides an apparatus for measuring temperature changes at or near one or more areas of a human subject, where the apparatus comprises a thermochromic pigment based garment. In one embodiment, the thermochromic pigment based garment of the apparatus is in the form selected from the group consisting of a bandage, a nonwoven bandage, a wearable strip, an adhesive wearable tape, and the like.

The present disclosure provides innovative advances in the field of functional apparel that can serve to monitor changes in temperature, and provides for the use of such apparel in indentifying fatigue or physical exhaustion in the person wearing the apparel, as well as for various other uses, including medical uses as well as athletic training and exercise uses. The present disclosure addresses the need for wearable garments or other wearable apparel products that can measure changes in temperature of a person for use in a variety of medical, athletic, and casual applications.

Therefore, in accordance with the present invention, the use of thermochromic pigments can enhance activewear by preventing exhaustion during athletic exercise while providing key desirable elements such as comfort and functionality. By employing thermochromic pigments that undergo a color change in the temperature range of elevated skin temperature during brisk exercise, the wearer of the garment can assess whether or not he is exercising too vigorously based on the garment panel colors. The present disclosure provides both a functional and fashionable designs with thermal sensing technology to create a performance athletic garment capable of signaling physical exhaustion to the wearer. As described herein, in certain aspects, the present disclosure provides a method for thermochromic pigment application to textiles, assessment of pigment performance, and design of novel apparel.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, as provided, like reference numerals contained in the drawings are meant to identify similar or identical elements.

The patent or application file contains at least one drawing executed in color. Copies of this patent or patent application publication with color drawings will be provided by the U.S. Patent and Trademark Office upon request and payment of the necessary fee.

FIGS. 1A-1B: Thermochromic pigment applied to Nylon/Spandex material exhibiting, (a) crocking of pigment coating on surface (FIG. 1A), (b) insufficient binding of pigment to textile surface (prominent in aqueous environments) (FIG. 1B).

FIGS. 2A-2B: Artistic renderings of apparel design for detection of physical exhaustion with varying thermochromic pigment colors and activation temperatures. The left of each figure illustrates the front view of the apparel design; the right of each figure illustrates the rear view of the apparel design. FIG. 2A: Illustrates one embodiment of thermochromic apparel having separate thermochromic temperature zones as follows: Black 35° C. zone; Blue 33° C. zone; Blue 38° C. zone; and Magenta 38° C. zone. FIG. 2B: Illustrates one embodiment of thermochromic apparel having separate thermochromic temperature zones, with the illustration being provided in black and white to show borders of the various thermochromic temperature zones, as well as non-thermochromic zones.

FIG. 3: Photo graphs comparing abrasion resistance of thermochromic coated Nylon/Spandex after (a) 0 cycles, (b) 10,000 cycles, and (c) 13,000 cycles.

FIG. 4: Graph illustrating Color Fastness of thermochromic coated Nylon/Spandex for control (untreated) and non-chlorine bleach treated (test) Black 35° C., Blue 38° C., and Magenta 38° C. fabrics. Values presented are an average of four measurements.

FIGS. 5A-5B: Photographs of one embodiment of a thermochromic apparel on a Thermoregulating Mannequin after 5 minute exposure to 35° C. (FIG. 5A) and 15 minute exposure (FIG. 5B). Insets: Blown up views of the arm coated with 35° C. black thermochromic pigment.

### DETAILED DESCRIPTION OF THE INVENTION

The present invention provides, inter alia, a thermochromic garment comprising a thermochromic pigment, a process for making a thermochromic garment comprising a thermochromic pigment, and methods for using a thermochromic garment comprising a thermochromic pigment, including, inter alia, methods involving monitoring changes in temperature of a human subject or a portion of the human subject that is in contact with the garment. The present invention also provides an apparatus for measuring temperature changes at or near one or more areas of a human subject, where the apparatus comprises a thermochromic pigment based garment.

As provided by the present disclosure, in certain embodiments, innovations such as thermochromic pigments can enhance activewear by preventing exhaustion during athletic exercise while providing key desirable elements such as comfort and functionality. By employing thermochromic pigments that undergo a color change in this temperature range, as provided in the present disclosure, the wearer of the garment can assess whether or not he is exercising too vigorously.

As provided by the present disclosure, looking more closely at this colorimetric technology, in certain embodiments, the thermochromic pigments are leuco dyes that have color changing abilities (Zhu 2005; Kulcar 2010). A thermochromic pigment has many components to it: 1) solvent, 2) weak acid, 3) salt of a fatty acid, and 4) leuco dye. When the temperature surrounding the pigments rises, the solvent present within the pigment microcapsules undergoes transition to liquid phase, after which the salt dissociates, which in turn lowers the pH within the capsule. With the salt acting as a proton donor, a ring on the leuco dye molecule becomes protonated, leading to opening of the ring structure. As a result, there is a significant shift in the absorption of the molecule, leading to a colorless appearance. In order to apply this technology to apparel design and fabrication, thermochromic pigments can be applied onto the surface of the textile material (Mather 2001; White 1999; Tang 2006), then used to create an overall athletic apparel piece. Depending on the temperature of the transition, the pigments can appear colored or colorless on the textile material.

The present disclosure relates, inter alia, to both a functional and fashionable design with thermal sensing technology to create a performance athletic garment capable of signaling physical exhaustion to the wearer. In the present disclosure, certain aspects that are disclosed include, without limitation, a method for thermochromic pigment application to textiles, assessment of pigment performance, and design of novel apparel. Through thermochromic apparel performance testing including abrasion resilience, color fastness, and functionality assessment, these materials indicate tremendous potential for uses in athletic applications to detect and reduce physical exhaustion.

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

### Process for Making a Thermochromic Garment

According to one aspect, the present disclosure provides a process for making a thermochromic garment. In one embodiment, the process comprises the steps of: (i) providing at least one thermochromic pigment-based coating solution comprising a unique thermochromic pigment having a desired activation temperature and activation color, a binding agent, and a thickener; (ii) applying the at least one thermochromic pigment-based coating solution to a target surface of a fabric at a uniform thickness to yield a coated fabric having at least one thermochromic zone comprising the unique thermochromic pigment; and (iii) curing the coated fabric using a heat treatment protocol effective to promote cross-linking, thereby yielding a thermochromic garment having at least one thermochromic zone.

In one exemplary embodiment, the at least one thermochromic pigment-based coating solution can include, without limitation, 4-10% of the unique thermochromic pigment, 18-20% of the binding agent, 10% of the thickener, and 70% of water. In a particular embodiment, the at least one thermochromic pigment-based coating solution is prepared by first providing the water and then mixing, in succession and while stiring vigorously, the binding agent, the unique thermochromic pigment, and then the thickener.

Various thermochromic pigments may be used in the process of the present invention. In embodiment, the unique thermochromic pigment comprises a leuco dye. Leuco dyes are described in more detail herein. In accordance to one embodiment, the unique thermochromic pigment used in the process can have an activation color selected from the group consisting of blue, black, red, magenta, yellow, purple, violet, orange, white, and shades thereof. In accordance with another embodiment, the unique thermochromic pigment can have an activation temperature selected from the group consisting of 31° C., 32° C., 33° C., 34° C., 35° C., 36° C., 37° C., 38° C., 39° C., and 40° C. In a more particular embodiment, the unique thermochromic pigment can have an activation color and an activation temperature corresponding to any combination of activation color and activation temperature, where the activation color is selected from the group consisting of blue, black, red, magenta, yellow, purple, violet, orange, white, and shades thereof, and wherein said activation temperature selected from the group consisting of 31° C., 32° C., 33° C., 34° C., 35° C., 36° C., 37° C., 38° C., 39° C., and 40° C.

Further, the unique thermochromic pigment is reversible in that its activation color will revert to a non-activated color state when its activation temperature is not maintained.

In accordance with one embodiment of the process of the present invention, the uniform thickness of the applied coating solution is a range selected from the group consisting of between about 0.1 millimeters (mm) and about 1.0 mm, between about 0.1 mm and about 0.8 mm, between about 0.1 mm and about 0.7 mm, between about 0.1 mm and about 0.6 mm, between about 0.1 mm and about 0.5 mm, between about 0.1 mm and about 0.4 mm, and between about 0.1 mm and about 0.3 mm.

The process for making the thermochromic garment of the present invention can further include the step of bleaching the fabric prior to applying the at least one thermochromic pigment-based coating solution to the target surface of the fabric, wherein said fabric comprises natural fibers (e.g., cotton). In a particular embodiment, the bleaching solution can include water (e.g., 30 times greater than the fabric weight), hydrogen peroxide, sodium hydroxide (NaOH), Clarite LTC, and Invadine. In one suitable example of this bleaching step, the fabric can be added to the bleaching solution and subjected to heating (e.g., on a hot plate), and then rinsed well with water, and thereafter placed on a flat surface to air dry.

The process for making the thermochromic garment of the present invention can also further include the step of scouring the fabric prior to applying the at least one thermochromic pigment-based coating solution to the target surface of the fabric. Methods of scouring fabric are known in the art. However, in one embodiment of the process of the present invention, the scouring step can include submerging the fabric in a scouring solution comprising water, sodium hydroxide (NaOH), Invadine, and Invadex. In a more particular embodiment, the process for making the thermochromic garment of the present invention can also further include the step bleaching the scoured fabric prior to applying the at least one thermochromic pigment-based coating solution to the target surface of the fabric, wherein said fabric comprises natural fibers (e.g., cotton).

As provided herein, the fabric used in the process can comprise natural fibers, synthetic fibers, or a combination thereof. In one embodiment, the fabric comprising natural fibers is cotton. In another embodiment, the fabric comprising synthetic fibers is selected from the group consisting of spandex, nylon, polyester, and combinations thereof.

In accordance with one embodiment of the process of the present invention, the heat treatment protocol of the curing step comprises, without limitation, drying the coated fabric at a temperature of about 100° C. and then further incubating the coated fabric at a temperature of about 160° C. The present invention contemplates the use of other heat treatment temperature that yield equivalent results as those temperatures specifically mentioned herein.

In accordance with one embodiment of the process of the present invention, during the applying step the fabric is subtly stretched to facilitate adherence of the thermochromic pigment to the fabric. Techniques for subtly stretching fabric for this purpose are described herein and known in the art.

The process for making the thermochromic garment can be used to prepare a variety of garments. For example, in one embodiment, the process involves providing a plurality of different thermochromic pigment-based coating solutions for applying to the fabric, where each thermochromic pigment-based coating solution comprises its own unique thermochromic pigment, and where each thermochromic pigment-based coating solution is applied to a different target surface of the same fabric or to its own designated piece of fabric for assembly into the thermochromic garment, thereby yielding a thermochromic garment comprising a plurality of different thermochromic zones.

In one embodiment, the process involves making the thermochromic garment so that it further comprises non-thermochromic zones, where such non-thermochromic zones do not include a thermochromic pigment. In a particular embodiment, the process yields a thermochromic garment where the non-thermochromic zone comprises a black colored or dark colored fabric and the thermochromic zones comprise white or substantially white fabric when the thermochromic pigments are not activated.

In one embodiment, the process involves making the thermochromic garment so that a plurality of different thermochromic zones can border or substantially border one another in order to provide a multi-part zone that exhibits a range of temperature activation.

In another embodiment, the process involves making the thermochromic garment so that a plurality of unique thermochromic pigments are applied to the same target surface of the fabric so as to provide a thermochromic zone that exhibits different pigment colors at different activation temperatures.

In a further embodiment, the process involves making the thermochromic garment so that the plurality of different thermochromic zones are located at regions corresponding to portions of a human selected from the group consisting of scalp, face, neck, upper back, lower back, shoulder, upper arm, tricep, inner arm, bicep, forearm, wrist, hand, waist, rear, groin, hip, upper thigh, lower thigh, inner thigh, calf, shin, achilles, foot, and portions thereof.

The uniqueness of the process for making the thermochromic garment is that it is able to produce garments that have superior abrasion and color fastness properties than other garments that are made with thermochromic pigments. For example, in accordance with one embodiment, the process of the present invention is such that the at least one thermochromic zone is durable in that the corresponding unique thermochromic pigment contained in the thermochromic zone remains active after abrasion testing up to about 10,000 cycles and maintains color fastness according to benchmark apparel testing standards. Methods for testing abrasion and color fastness are described herein and known in the relevant field of art.

In accordance with one embodiment of the process described herein, the process comprises integrating the thermochromic pigment to or into a fiber or textile precursor of the fabric prior to producing the thermochromic garment. The fiber or textile can be any fiber or textile used to make any garment suitable for wearable use by a human.

In accordance with one embodiment of the process, the thermochromic garment can be made of any fabric, fiber, or textile suitable for use as a wearable garment for a human, including, but not limited to, fabrics, fibers, or textiles used for conventional garments.

In accordance with another embodiment of the process, the thermochromic garment can be a type of garment selected from the group consisting of athletic wear, active wear, sportswear, casual wear, medical clothing, functional wear, underwear, and the like.

In accordance with a further embodiment of the process, the thermochromic garment can be in the form of a garment selected from the group consisting of a shirt, shorts, pants, a sock, a glove, a headband, headwear (e.g., skull cap, hat, etc.), neckwear, a wrist band, an undergarment (e.g., upper body or lower body undergarments), compression wear (e.g., compression shorts, compression pants, compression stockings, compression torso wear, etc.), a sleeve (e.g., arm sleeve, leg sleeve, etc.), a wrap (e.g., ankle wrap, knee wrap, neck wrap, foot wrap, hand wrap, arm wrap, wrist wrap, leg wrap, etc.), and the like.

In another embodiment of the process, the process comprises integrating the thermochromic pigment to or into a fiber or textile precursor prior to producing the garment. The fiber or textile can be any fiber or textile used to make any garment suitable for wearable use by a human.

In one aspect, the present invention also is directed to a thermochromic garment produced by the process as described above and as further illustrated in the drawings and Examples.

### Thermochromic Garment

According to one aspect, the present disclosure provides a thermochromic garment. In one embodiment, the thermochromic garment comprises a fabric having a plurality of thermochromic zones and at least one non-thermochromic zone. As used herein, each thermochromic zone comprises a thermochromic pigment having a desired activation temperature and a desired activation color. As used herein, the at least one non-thermochromic zone is defined in that it does not comprise a thermochromic pigment.

In one embodiment, the thermochromic pigment of the thermochromic garment comprises any pigment that turns color in response to a change in temperature. More particularly, the thermochromic pigment of the garment can include, without limitation, a leuco dye. Suitable thermochromic pigments can have an activation color selected from the group consisting of blue, black, red, magenta, yellow, purple, violet, orange, white, and shades thereof. Further, suitable thermochromic pigments can have an activation temperature selected from the group consisting of 31° C., 32° C., 33° C., 34° C., 35° C., 36° C., 37° C., 38° C., 39° C., and 40° C. In other embodiments, a suitable thermochromic pigment can have an activation color and an activation temperature corresponding to any combination of activation color and activation temperature, wherein said activation color is selected from the group consisting of blue, black, red, magenta, yellow, purple, violet, orange, white, and shades thereof, and wherein said activation temperature selected from the group consisting of 31° C., 32° C., 33° C., 34° C., 35° C., 36° C., 37° C., 38° C., 39° C., and 40° C.

A suitable thermochromic pigment can be one that is reversible in that its activation color will revert to a non-activated color state when its activation temperature is not maintained.

In accordance with one embodiment of the thermochromic garment of the present invention, the thermochromic pigment is on a surface of the fabric at a uniform thickness in a range selected from the group consisting of between about 0.1 millimeters (mm) and about 1.0 mm, between about 0.1 mm and about 0.8 mm, between about 0.1 mm and about 0.7 mm, between about 0.1 mm and about 0.6 mm, between about 0.1 mm and about 0.5 mm, between about 0.1 mm and about 0.4 mm, and between about 0.1 mm and about 0.3 mm.

The thermochromic garment can be such that the fabric comprises natural fibers, synthetic fibers, or a combination thereof. In one embodiment, the fabric comprising natural fibers is cotton. In another embodiment, the fabric comprising synthetic fibers is selected from the group consisting of spandex, nylon, polyester, and combinations thereof.

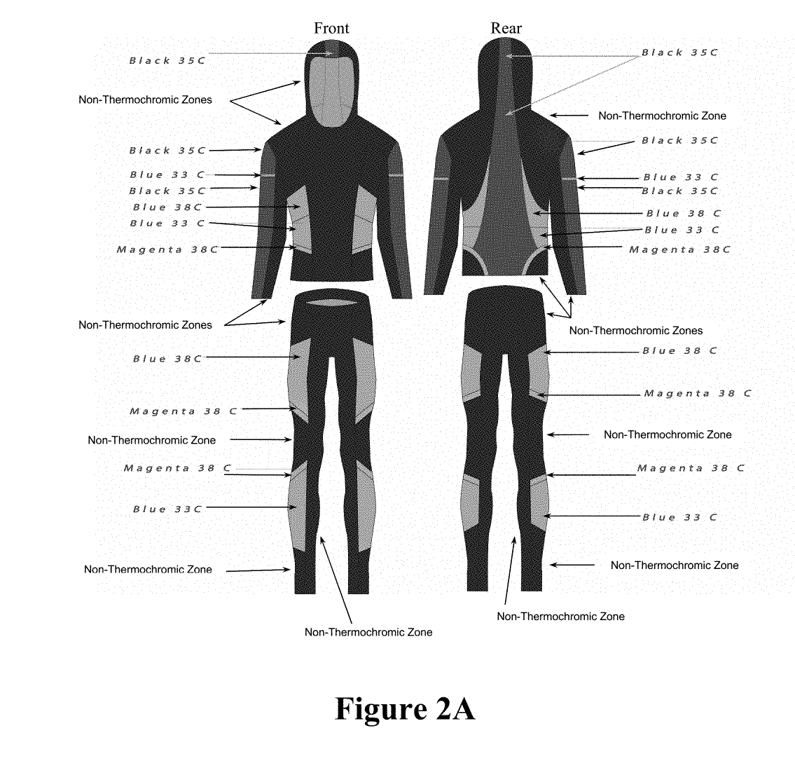

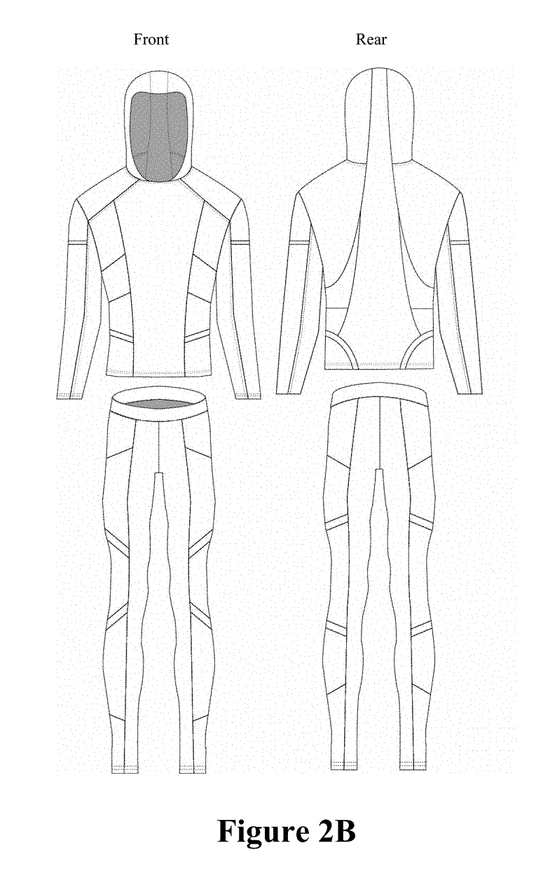

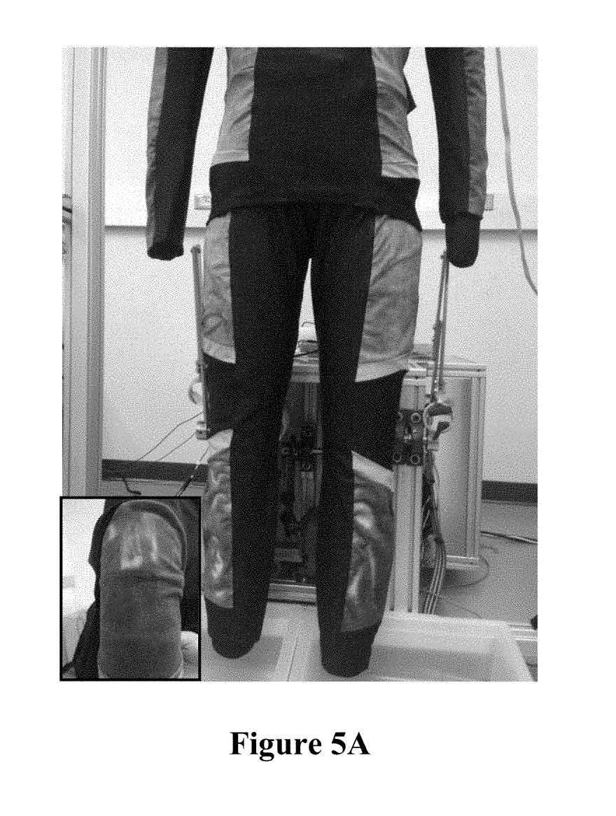

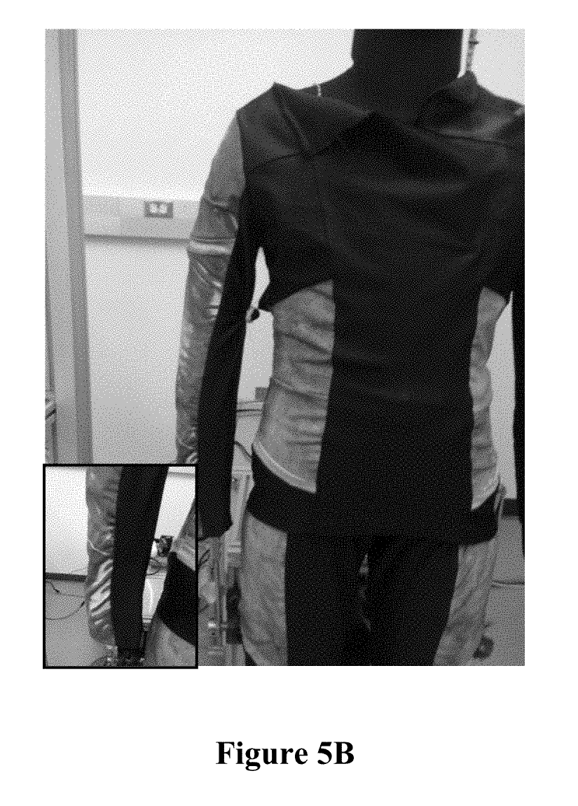

In one embodiment, the thermochromic garment further comprises non-thermochromic zones, said non-thermochromic zones not comprising a thermochromic pigment. As used herein, the non-thermochromic zone can comprise a black colored or dark colored fabric and the thermochromic zones can comprise white or substantially white fabric when the thermochromic pigments are not activated. The illustrations of FIGS. 2A-2B and the photographs of FIGS. 5A-5B show embodiments thermochromic garments of the present invention having a plurality of thermochromic zones and a plurality of non-thermochromic zones.

In a particular embodiment, the thermochromic garment is such that a plurality of different thermochromic zones can border or substantially border one another in order to provide a multi-part zone that exhibits a range of temperature activation.

In another embodiment, the thermochromic garment is such that a plurality of unique thermochromic pigments is contained on a target surface of the fabric so as to provide a thermochromic zone that exhibits different pigment colors at different activation temperatures.

In a further embodiment, the thermochromic garment is such that the plurality of different thermochromic zones are located at regions corresponding to portions of a human selected from the group consisting of scalp, face, neck, upper back, lower back, shoulder, upper arm, tricep, inner arm, bicep, forearm, wrist, hand, waist, rear, groin, hip, upper thigh, lower thigh, inner thigh, calf, shin, achilles, foot, and portions thereof.

The thermochromic garment of the present invention is such that the at least one thermochromic zone is durable in that the corresponding unique thermochromic pigment contained in the thermochromic zone remains active after abrasion testing up to about 10,000 cycles and maintains color fastness according to benchmark apparel testing standards.

In one embodiment, the thermochromic garment is made of any fabric, fiber, or textile suitable for use as a wearable garment for a human, including, but not limited to, fabrics, fibers, or textiles used for conventional garments.

In another embodiment, the thermochromic garment is a type of garment selected from the group consisting of athletic wear, active wear, sportswear, casual wear, medical clothing, functional wear, underwear, and the like.

In a further embodiment, the thermochromic garment is in the form of garment selected from the group consisting of a shirt, shorts, pants, a sock, a glove, a headband, headwear (e.g., skull cap, hat, etc.), neckwear, a wrist band, an undergarment (e.g., upper body or lower body undergarments), compression wear (e.g., compression shorts, compression pants, compression stockings, compression torso wear, etc.), a sleeve (e.g., arm sleeve, leg sleeve, etc.), a wrap (e.g., ankle wrap, knee wrap, neck wrap, foot wrap, hand wrap, arm wrap, wrist wrap, leg wrap, etc.), and the like.

In general, for thermochromic apparel of the present disclosure, the family of leuco dyes can be the primary type of chemical used. They are used for this application due to their inherent chemical structure. These molecules change color based on their “activation” temperature. In essence, as specific temperatures (e.g., temperatures near physiological temperatures), the chemistry of the dye within the pigment sphere changes. The ring of the dye opens after hydrogen protonation and the absorbance of the molecule changes from visible to colorless. The leuco dyes (which are used for color chromism) are the main color change component of the 4-component pigment sphere. The other components include the resin (for the outside of the sphere), solvent (which melts at the activation temperature), and proton donor system (fatty acid).

Suitable leuco dyes are as described herein above and below, and can include, without limitation, leuco dyes provided by Kelly Chemical Corporation (Taipei, Taiwan) under the product names such as Thermochromic Pigment (Blue), Thermochromic Pigment (Red), and Thermochromic Pigment (Black). Other leuco dyes can include, without limitation, those described in the following Internet links: www.karlssonrobotics.com/cart/thermochromatic-pigment-black-20g/?gclid=CO-HmPrbwMECFQ9p7AodJT4ATA; and www.amazon.com/Thermochromic-Pigment-Color-Pods-Activated/dp/B00FFFONRG/ref=sr_1_sc_1?ie=UTF8&qid=1413996675 &sr=8-1-spell&keywords=thermochromic+pigmetns.

### Methods of Using a Thermochromic Garment

According to another aspect, the present disclosure provides various methods for using a thermochromic garment described herein.

In one embodiment, the method involves monitoring changes in temperature of a human subject or of a portion of the human subject. This method involves the steps of: (i) providing a thermochromic garment according to any one of claims 34-52 to a human subject; and (ii) monitoring changes in color of the thermochromic zones of the thermochromic garment while the human subject wears said thermochromic garment, wherein a change in the color of a thermochromic zone indicates a corresponding change in the temperature of the human subject or in a portion of the human subject.

In one embodiment, the method relates to monitoring or treating various conditions or aspects of the human subject selected from the group consisting of physical exhaustion, diabetes, arthritis, general ulcers (e.g., skin ulcers), pressure ulcers, and changes in temperature of portions of the human subject.

Any of the embodiments of the thermochromic garment described herein are suitable for use in the methods of use of the present invention.

Various embodiments of the use of the thermochromic pigment based garments and apparel are described in more detail below.

Endurance athletes face a complex array of physiological changes as their core and skin temperature rises in vigorous exercise. In a particular embodiment, to aid in facile and accurate determination of physical exhaustion, thermochromic pigments were applied to Nylon/Spandex fabric, using pigment activation temperature as an indication for exhaustion. As provided herein, thermochromic pigments contain leuco dyes capable of changing chemical structure to alter the dye molecule absorbance, leading to a visual tool for skin temperature indication. This technology was successfully coupled with conventional textiles to create smart apparel with excellent abrasion and color fastness capabilities. Using targeted placement of thermochromic panels in garment construction, a garment capable of serving as a “warning light” for physical exhaustion in athletes was successfully created.

The methods of use of the present disclosure include any use of a thermochromic pigment based garment or apparel product involving monitoring changes in temperature of the wearer of the garment. Certain particular applications for using the thermochromic pigments in garments, apparel, and apparatuses are further described below.

Diabetic Socks: Diabetics have increased risk of foot ulcers. Skin temperature rises before ulcer formation. A sock coated with thermochromic pigments can serve as a warning for the formation of (venous) ulcers (see, e.g., Armstrong et al. 2007).

Arthritis Wrist Band/Sock: These socks will detect inflammation that is inherent in the disease, and will indicate excess inflammation in joints. See, e.g., Collins et al. 1974; Horvath et al. 1948; Jeschonneck et al. 2000; and Korn et al. 2002.

General Ulcers for Patients in Hospitals: At doctors' discretion, a physician could have a patient wear a thermochromic sock, ankle band, wrist band, etc., to serve as a warning for the formation of ulcers.

Medical Bandages (e.g., nonwovens): For patients who need medical bandages for long-term injuries, thermochromic bandages can alert a patient/physician if the area is infected.

Pressure Ulcers in Neurologically Impaired Patients: According to a reported study (Sae-Sia et al 2005), 7.6-36% of neurologically impaired patients develop pressure ulcers. Skin temperature rises before a pressure ulcer occurs. These ulcers are commonly found around the elbows, knees, and ankles. Thermochromically coated ankle, knee, or elbow braces could be used as a warning of formation of these ulcers.

According to another aspect, the present disclosure provides an apparatus for measuring temperature changes at or near one or more areas of a human subject, where the apparatus comprises a thermochromic pigment based garment. In one embodiment, the thermochromic pigment based garment of the apparatus is in the form selected from the group consisting of a bandage, a nonwoven bandage, a wearable strip, an adhesive wearable tape, and the like. As provided herein, the thermochromic pigments described and/or contemplated herein are suitable for use in the apparatus of the present invention.

### EXAMPLES

The following examples are intended to illustrate particular embodiments of the present invention, but are by no means intended to limit the scope of the present invention. While illustrated in the Examples section, below, those of ordinary skill in the art will recognize that the instant disclosure enables similar compositions to be developed from alternative starting materials and intermediates.

### Example 1

### Development of Thermochromic Pigment Based Sportswear for Detection of Physical Exhaustion

Endurance athletes face a complex array of physiological changes as their core and skin temperature rises in vigorous exercise. Since endurance athletes do not typically have fiscally feasible and straightfoward real time physiological monitoring outside of complex Bluetooth and wireless connectivity linking their heart rate, pulse oximetry, etc. to electronic databases, exercise reaching high levels of physical exhaustion is of great concern. To aid in facile and visual determination of physical exhaustion, thermochromic pigments were applied to Nylon/Spandex fabric, using pigment activation temperature as an indication for exhaustion. Thermochromic pigments were chosen because these microcapsules contain leuco dyes capable of changing chemical structure to alter the dye molecule absorbance, leading to a visual tool for skin temperature indication. Each pigment was chosen to activate at a targeted physiological skin temperature range between 33-38° C. Nylon/Spandex was chosen for high wearer comfort and excellent garment/skin contact. This technology was coupled with conventional textiles to create smart apparel with satisfactory abrasion and color fastness capabilities. Using targeted placement of thermochromic panels in garment construction, a garment capable of serving as a “warning light” for physical exhaustion in athletes was created.

### Execution of Design: Material Preparation and Prototype Production

Thermochromic pigments of various activation temperatures (black 35° C., blue 33° C., 38° C., and magenta 38° C.), scouring reagents (Invatex AC-US (poly maleic acid), Invadine DA (alcohol C13-C15 poly(1,6) ethoxylate)), binding agent (Lyoprint PBA (poly butyl acrylate)) and thickening agent (Lutexal, First Source Worldwide) were used. Sodium hydroxide (NaOH) pellets (Mallinckrodt Chemicals, Phillipsburg, N.J., USA) were used for fabric scouring. White 87% Nylon/13% Spandex fabric (Spandex World Inc., New York, N.Y., USA) was used for thermochromic coating and black 87% Nylon/13% Spandex fabric (Spandex World Inc., New York, N.Y., USA) was used for the remainder of the garment.

### Scouring the Fabric:

Tap water was placed in 1000 ml beaker (30× total weight of fabric) with 50 g/L NaOH, 3 g/L Invadine DA, and 8 g/L Invadex AC-US. Once the solution reached a rolling boil, Nylon/Spandex fabric was added with the temperature lowered to a simmer for 30 minutes. The hot scouring solution was carefully decanted and the fabric rinsed well (10×) in tap water. The scoured fabric was then dried at ambient room temperature on a flat surface.

### Pigment Solutions and Application to Fabric:

Pigment solutions were created using the following composition; 4-10% pigment, 18-20% binding agent, 10% thickener, and 70% water. For example, 7 g of water was added to a small beaker, followed by 2 g binder. Next, 0.4 g pigment was added. Lastly, 1 g thickener was placed in the beaker. Upon vigorous stirring, the solution became noticeably viscous. In order to balance wearer comfort with the most striking color change effect, the weight percent of pigment in solution was varied, starting with the least amount to give good effect (˜4 wt %). At this pigment feed ratio, color change was well recognizable and reversible, yet overall appearance of color on the fabric was medium/light. For example, at a total solution concentration of 4 wt %, blue 33° C. thermochromic pigments were reasonably colored, but lacked a striking depth of color on the surface. To increase the color saturation of thermochromic pigments on the fabric, the weight percent of pigment in solution was increased to ˜10%, and the overall effect was a definitive intensification in color saturation with acceptable textile surface texture. The result of increasing the weight percent of thermochromic pigments in the solution slurry increased the color depth (in the case of blue 33° C.) from medium blue with hints of white base textile to rich royal blue with no visibility of spandex fabric.

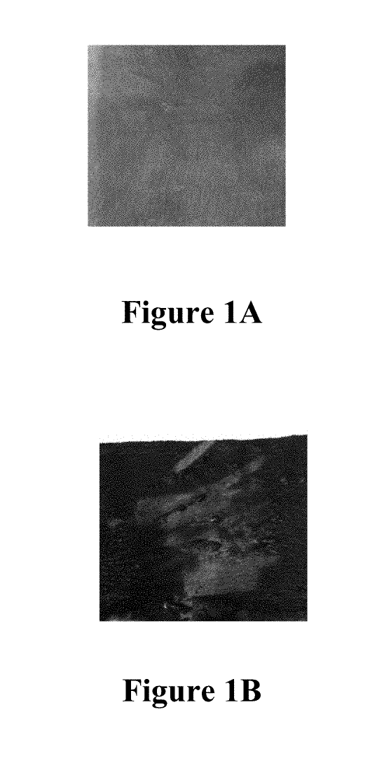

Nylon/Spandex was cut to the appropriate size. Once the fabric was secured well to a flat surface, a brush was used to apply the viscous pigment solution to the surface of the fabric using even brush strokes. When applying pigment to surface of textile, it was imperative to place a thin but consistent layer. Too thick a layer caused bulk pigment on the surface, leaving an uneven patchy appearance that over time excess started to peel away (FIG. 1A) (i.e., crocking). Furthermore, experiments showed that using an extremely thick coating layer caused ineffective crosslinking of the binder. The resultant effect was removed of the pigment coating upon immersion in an aqueous environment (FIG. 1B).

To mitigate the difficulty with ineffective binder, experimental results indicated that applying a consistent thin coat with subtle stretching of the fabric facilitated good pigment adherence. Coated fabric pieces were dried in an isotemperature oven at 100° C. for 10 minutes or until completely dry. The fabric was then removed while the oven heated to 160° C. Once at temperature, the fabric was placed back in the oven and crosslinked for approximately 5-7 minutes. Care was taken in the pigment application process to minimize exposure of pigment treated fabric to high temperatures.

### Abrasion Characterization

In order to better understand how durable thermochromic apparel would be for repeated wear, abrasion testing was used. The testing done was based on ASTM D4966-12-Standard Test Method for Abrasion Resistance of Textile Fabrics (Martindale Abrasion Tester Method) with minor modifications (Blau 1999). A 5.5 inch circular sample of fabric (coated with blue 38° C. thermochromic pigment) was cut on a piece of felt with a circular cutter. The samples were placed on the abrasion tester and were rubbed against the standard test fabric (jersey knit). The samples were first observed for every 100 cycles to observe how the appearance of the fabric changed (noting any color differences or formation of broken yarns, holes, or pilling). Abrasion testing was done until the fabric was subject to 2000 cycles. The samples were then checked every 500 cycles until the fabric was subject to 10,000 rubs. Finally, the fabric was then checked every 1500 cycles until the fabric reached 13,000 rubs.

### Color Fastness Characterization

As athletes set constant and vigorous workout schedules, activewear must be able to withstand laundering while maintaining color fastness. To quantify color fastness of thermochromic textiles, a mechanical colorimeter was used to characterize adherence of the thermochromic pigment to the fabric. In addition to the thermochromic fabric retaining its functional capabilities, it also must retain its color for aesthetic purposes. One factor that can influence the color fastness of the garment is the molecular structure of the pigment (or dye); this in turn can affect the interaction of the pigment with the fabric. Material characteristics can impact the color fastness including the size (diameter) of the thermochromic microsphere are the size of the particle (Leelajariyakul 2008).

External factors such as UV and chlorine bleach can also influence the color fastness of thermochromic apparel. Therefore, the durability of the thermochromic pigment against non-chlorine bleach was assessed (The fabric was not subjected to chlorine bleach due to potential permanent damage to the thermochromic pigments). The results from color fastness testing dictate the instructions on a care label (Fan 2009). To assess the performance against non-chlorine bleach, the fabric was subjected a modified version of the AATCC 188 Test: Color Fastness to Sodium Hypochlorite Bleach in Home Laundering. The fabric changes in color were quantified via L (lightness to darknesss), A (red to green), and B (blue to yellow) respectively.

The testing done was based on AATCC Test Method 172—Quick Method for Color fastness to Non-Chlorine Bleach (Easter 2006). In brief, fabric coated with thermochromic pigment was cut into 3″×3″ squares. Non-chlorine bleach (peroxide-based laundry agent) was applied to the fabric pieces (denoted treated) and allowed to sit at room temperature for 5 minutes. Control and treated thermochromic fabric swatches were tested for color fastness using Macbeth Color Eye Colorimeter (X-Rite Ltd., Grand Rapids, Mich., USA). An average of four independent measurements were measured with L (lightness to dark scale), A (red to green), and B (blue to green) values recorded. ΔE values for color fastness were calculated using Equation (1).

ΔE*_{ab}=\sqrt{(L*₂−L*₁)²+(a*₂−a*₁)²+(b*₂−b*₁)²} (Equation 1)

Where a: represents the difference between green (a*₂) and red (a*₁), b: represents the difference between blue (b*₂) and yellow (b*₁) and L: represents the difference between black (L*₂) and white (L*1). This equation was used to assess the scale of color difference between two samples.

### Garment Design and Construction

The design of the garment integrated both the aesthetic design of athletic apparel and the function of the thermochromic pigments. Fabric was bought from a spandex retailer, chosen for fiber content, optimal thickness, and a matte finish. The fabrics chosen were black and white 87% nylon/13% spandex knits. Hydrogen bonding of polyamide chains within the nylon contributes to the mechanical properties of high modulus, stiffness, abrasion resistance, and low friction coefficient. These properties reduce wear-and-tear of the fabric, while also increasing tactile comfort. Spandex was needed to allow the stretch for a compressive garment, while also having dimensional stability after deformation. The high percentage of Spandex brings much more elongation to the fabric properties than a high modulus. The thick material followed the functionality of the clothing for outdoor running, while the matte finish offered both enhanced aesthetics and feasibility of construction. The white was chosen to apply the pigment to while the black was for the remainder of the garment. A two-piece tight fitting compression ensemble, long sleeved hooded top and running pants was sketched to have maximum contact to the skin and pigments.

The design lines on the hooded top emphasize an ideal muscular structure for a male. Pattern pieces on both sides of the torso highlight abdominal muscles. Additionally, the center back pattern piece of the top resembles the extension of the spine, leading up to the hood while design lines on the back follow shoulder blade curvature. Both garments relied on the stretch of the fiber in the hooded top or the waistband in the pants for easy dressing and undressing.

The design considerations for the pants followed those of the hooded top. The segmented pieces on the outer leg of the pants were constructed to highlight thigh and calve muscular structure. Lines from the geometric pattern pieces are horizontally continuous around side seams, and vertically continuous from top to bottom creating interesting views from all angles.

Pigments were assigned to pattern pieces of the garment by looking at skin temperatures relating to extreme core body temperatures. The black and coated white pieces were sewn together. Thermochromically-coated apparel pieces were integrated into the design to highlight physiological areas that generate heat through the skin during rigorous exercise. For example, in the upper body piece, the sleeve inserts are coated with black 35° C. pigments, while the core inserts are coated with blue 33° C. and magenta 38° C. (FIGS. 2A-2B).

To best show a range of temperature as core and skin temperature increases, the upper thigh is coated with blue 38° C., followed by magenta 38° C., ending with blue 33° C. Pigments having an activation temperature around 38° C. are crucial as this is on the upper limit of skin temperature for physical exhaustion. Thus, when thermochromically coated inserts in the upper thigh change from colored to colorless, the athlete has a quick, effective, and visual method for determining when rest is needed to maintain healthy cardiac output.

### Evaluation of Effects of Thermochromic Pigment Using a Sweating Thermal Manikin

The garment was tested on Walter® (Hong Kong Polytech University, Hong Kong), a sweating thermal manikin. Following ISO11097 (Ergonomics of the thermal environment, BS EN ISO 11079; 2007 standard), the mean skin temperature of a sweating thermal manikin was controlled at 35° C. to simulate mean skin temperature of human body. Environmental chamber remained at 21° C.±2°/65%±5% relative humidity. Qualitative observations were recorded for garment panel color change as a function of time from the program Walter®—Sweating Fabric Manikin System 2012. Qualitative observations included photographically documenting the amount of time after placing the garment on the manikin for each colored pigment piece to activate based on the manikin surface temperature.

### Results and Discussion

### Pigment Application

Pigment solutions were created using the aforementioned composition. It was found that the order in which each component was added in succession affected the overall uniformity of the mixture; adding the water first, followed by binder, pigment, with thickener last afforded the best solution. Mixing the pigment with water before addition of binder caused the powder to become unmanageable; the pigment spheres would not integrate well into solution and the resulting slurry became uneven and patchy in distribution. Applying a thin consistent layer of thermochromics pigment slurry to the fabric surface afforded an even coating, good color saturation, and reversible color change. A general schematic of the overall garment is given in FIGS. 2A-2B.

### Garment Performance

Due to the stringent and high impact wear of athletic apparel, it was necessary to characterize garment performance using standard textile quantification protocols. In order to ensure acceptable durability and quality of the bodysuit made with thermochromics pigments, it was necessary to assess abrasion resistance, color fastness, and pigment activation sensitivity. Because dynamic body movements are likely to cause abrasion leading to degradation of the surface thermochromics pigment coating, it was paramount to determine how robust the coating was to surface agitation. Furthermore, since athletic apparel must be stable when subject to repeated washing, color fastness was an imperative parameter to examine to determine durability and stability to common detergent. Lastly, testing on a thermal manikin was used to confirm that pigment color change in a physiologically relevant system (i.e. mimicking normal and elevated skin temperature) was observed for each thermochromic activation temperature, indicating appropriate color change would occur while the user was wearing the garment to ensure proper function.

### Abrasion Analysis

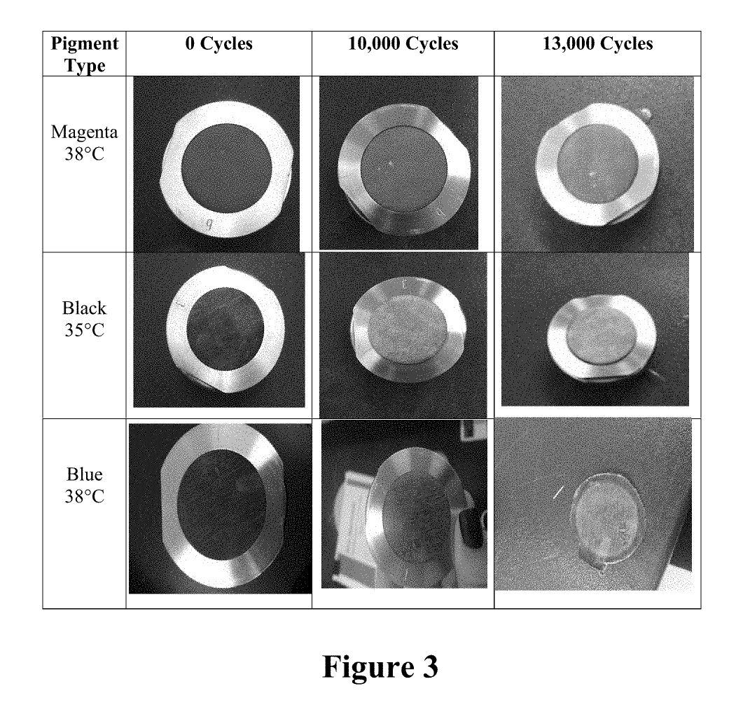

Abrasion testing was used to assess the overall durability of thermochromic coating on the surface of the fabric. It was noted that some pilling and broken yarns began to form (minimally) at around 1400 cycles. Nonetheless, the pigment remained intact on the fabric. Thermochromic activity of the fabric at the activation temperature was still observed after 10,000 cycles (FIG. 3). Once the fabric reached 13,000 cycles, it was difficult to detect the thermochromic capabilities of the fabric unless one applied forced heat on the fabric (FIG. 3). The fabric utilized for the garments were purchased from Spandex World Inc., a company specializing in spandex blends for all functions. The fiber content of fabric used for thermochromic apparel was 87% nylon and 13% percent spandex, relatively low modulus and highly elastic fibers. In reference to ASTM D4970-Standard Test Method for Pilling Resistance and Other Related Surface Changes, this fabric received a rating of 4 after of 13,000 cycles, indicating slight surface fuzzing.

### Color Fastness Analysis

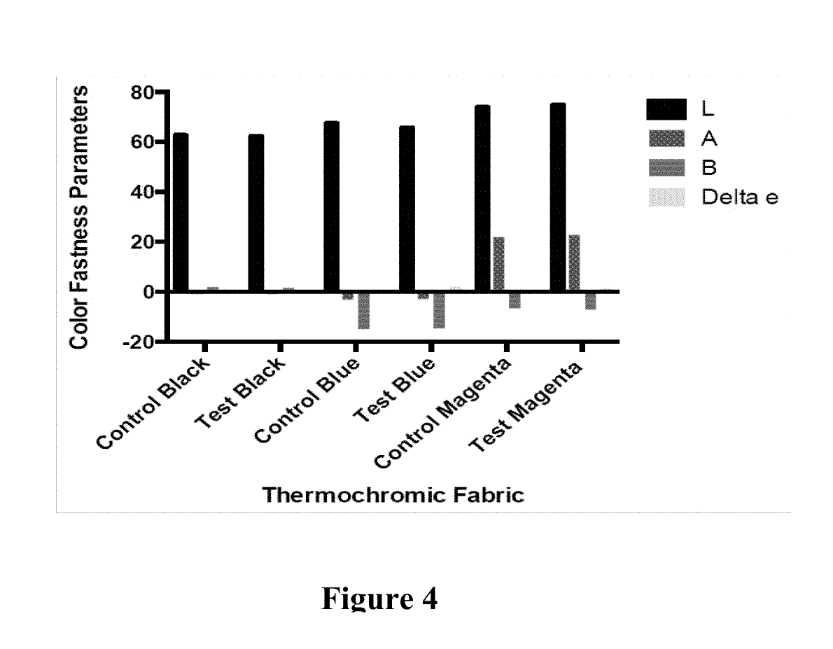

The ranking in terms of greatest to least color change (as quantified by ΔE) were blue 33° C., magenta 38° C., and black 35° C. (FIG. 4); the ΔE values were 1.966, 1.427, 0.467 respectively (calculated using Eq. 1). Due to limited sample, each thermochromic was tested singularly. According to the AATCC standards, if the ΔE value is less than 2.0, there is no considerable difference in color change between the sample types. All thermochromic coated apparel types met this benchmark, demonstrating the durability of the fabric against non-chlorine bleaches, indicating sufficient wash resistance. In addition to assessing the impact of the bleach on the fabric, it was observed that the greater abrasion of the surface lead to increased crocking of the pigment of the surface. The increased crocking was expected to reveal a greater change in color and ΔE value.

### Thermoregulating Manikin

After 5 minutes being placed at 35° C. the thermal manikin, color change was visually observed using qualitative photographs. The 33° C. blue panels on the garment showed only minimal color change while pieces coated with 35° C. black showed no visible color change (FIG. 5A). The locations of prominent color change were observed primarily in the lower calf region and arm inserts where the largest panel of blue 33° C. were integrated into the garment.

After 15 minutes of donning of the prototype garment on the thermal manikin, black 35° C. panels located on the sleeves arms activated (FIG. 5B), along with stark color change in the blue 33° C. panels along the calf and arm side inserts. The 38° C. magenta and 38° C. coated panels did not change color throughout the heating process. The higher activation temperature magenta and blue 38° C. thermochromic panels were not expected to change color as the activation temperature was higher than the threshold of the manikin (summary given in Table 1).

TABLE 1
Summary of Thermochromic Pigment
Textile Performance Parameters

| | Total wt % of | Color Fastness | ΔT for Color |
|---|---|---|---|
| Pigment Type | Pigment | (ΔE)* | Change on Manikin |
| Magenta 38° C. | 10 | 1.18 ± 0.9 | <5 mins |
| Black 35° C. | 10 | 3.94 ± 0.31 | 5-10 mins |
| Blue 38° C. | 10 | 0.84 ± 0.71 | Did not change |

Use of the thermal manikin demonstrated that each targeted panel changed color at elevated skin temperatures in an environment similar to human use, proving our methodology for assigning the pigments.

### CONCLUSIONS

This Example presented a facile method for preparing and constructing athletic apparel that specifically targets exhaustion during workouts and can serve a functional purpose of visually signalling physical exhaustion via changes in apparel color caused by increasing skin temperature. Application of thermochromic pigments through surface treatment caused unexpected construction problems. The pigments were found to crock around areas pierced with the needle when sewing, such as seams and hems, and were further agitated when the fabric was stretched while compressed over the model. The coated thermochromic pieces of fabric were scoured, causing the fabric to naturally curl, and was harder to sew seams evenly with untreated fabric due to the thickness difference. Looking toward future optimization, in order to alleviate difficulties in construction, innovative techniques could be used. Adhesives may be used to make seams in future garments with surface treatments of thermochromic materials to eliminate the need to use a sewing machine. Thermochromic pigments could also be applied as a thinner layer by stretching the fabric, which would help the issue of crocking, and also, maintain properties, such as thickness, between treated and untreated fabrics. The possibility of applying a finish after surface treatment of thermochromic pigment could also increase durability and decrease the curling of the fabric.

The thermal manikin provided a controlled environment to confirm the pigment color change. While the thermal manikin simulated average skin temperature based on ISO standards and mimicked elevated skin temperature, this approach was limited as the thermal manikin was stationary and the test garments were designed for athletes in motion. Thus, future studies should include human performance testing to confirm the results found in this study. Additionally, human performance testing would provide information regarding the comfort of the garments and the experience of the wearer.

This study was the exploration of applying a functional pigment to parts of the surface of designed athletic apparel. Further studies would include the optimization of applying pigment to the varying fiber types, and how the specific piece of athletic apparel would undergo stress on the fabric, surface of fabric, and seams. Although future optimization is needed to improve garment construction and longevity, preliminary testing indicates our thermochromic apparel meets many of the key needs of smart apparel including maintainability, durability, usability in the field, and most importantly, functionality through abrasion resilience, color fastness, and color activation verification using skin temperature modeling.

### REFERENCES

Citation of a reference herein shall not be construed as an admission that such reference is prior art to the present invention. All references cited herein are hereby incorporated by reference in their entirety. Below is a listing of various references cited herein:
[1] Blau, P. J., K. G. Budinski., Development and Use of ASTM Standards for Wear Testing, Wear. 225-229 (1999) 1159-170.
[2] Branson, D., P. Kamenidis, S. Peksoz, H. Park, S. K. An, C. Stan, Proc. of The 8th International Meeting Manikins and Modeling, Victoria, BC (2010).
[3] Easter, E. P., B. E. Ankenman, Evaluation of the care and performance of comfort-stretch knit fabrics, AATCC Review. 6.11 (2006) 28-32.
[4] Ergonomics of the Thermal Environment. Determination and Interpretation of Cold Stress When Using Required Clothing Insulation (IREQ) and Local Cooling Effects. London: BSI, 2008.
[5] Fan, J., and L. Hunter. Engineering Apparel Fabrics and Garments. Cambridge, U.K.: Woodhead, 2009
[6] Fowler, D., The attributes sought in sports apparel: A ranking, J. Marketing Theory and Practice. 7.4 (1999) 81-88.
[7] Gonzalez-Alonso, J., R. Mora-Rodríguez, P. Below, E. Coyle., Dehydration markedly impairs cardiovascular functionin hyperthermic endurance athletes during exercise, J. Appl. Physiol. 82.4 (1997) 1229-36.
[8] Gonzalez-Alonso, J., C. Teller, S. Anderson, F. Jenson, T. Hyldig, B. Nielson, Influence of body temperature on the development of fatigue during prolonged exercise in the heat, J. Appl. Physiol. 86.3 (1999) 1032-39.
[9] Kul\check{c}ar, R., M. Fri\check{s}kovec, N. Hauptman, A. Vesel, M. Klanj\check{s}ek Gunde, Colorimetric properties of reversible thermochromic printing inks, Dyes Pigm. 86.3 (2010) 271-277.
[10] Leelajariyakul S., H. Noguchi, S. Kiatkamjornwong, Surface modified and micro-encapsulated pigmented inks for inkjet printing on textile fabrics, Prog. Org. Coat. 62.2 (2008) 145-61.
[11] Mather, R. R., Intelligent Textiles, Color. Tech. 31.1 (2001) 36-41.
[12] Mitsubayashi, K., Y. Wakabayashi, D. Murotomi, T. Yamada, T. Kawase, S. Iwagaki, and I. Karube, Wearable and Flexible Oxygen Sensor for Transcutaneous Oxygen Monitoring. Sensors and Actuators B: Chemical 95.1-3 (2003) 373-77.
[13] Morris, D., S. Coyle, Y. Wu, K. T. Lau, G. Wallace, and D. Diamond, Bio-sensing Textile Based Patch with Integrated Optical Detection System for Sweat Monitoring. Sensors and Actuators B: Chemical 139.1 (2009) 231-36.
[14] Mukhopadhyay, A., and Vinay Kumar Midha, A Review on Designing the Waterproof Breathable Fabrics Part II: Construction and Suitability of Breathable Fabrics for Different Uses, J. Ind. Tex. 38.1 (2008)17-41.
[15] Park, H., An, S. K., Peksoz, S., Cao, H., & Branson, D. Core Body Temperature Prediction through Monitoring of Microclimate under Body Armor Using Thermal Manikin, AATCC Review. 12.2 (2012) 69-72.
[16] Park, S., S. Jayaraman, Enhancing the Quality of Life through Wearable Technology, IEEE Engineering in Medicine and Biology Magazine. 22.3 (2003) 41-48.
[17] Peksoz, S., H. Cao, H. Park, S. K. An, and D. Branson. Proc. of the 8th International Meeting Manikins and Modeling, Victoria, BC (2010).
[18] Savastano, D. M., A. M. Gorbach, H. S. Eden, S. M. Brady, J. C. Reynolds, J. A. Yanovski, Adiposity and human regional body temperature., Am. J. of Clinical Nutrition. 90.5 (2009) 1124-31.
[19] Tang, S., L. Po, G. K. Stylios, An overview of smart technologies for clothing design and engineering, Int. J. of Clothing Science and Technology, 18.2 (2006) 108-128.
[20] White, M. A., M. Leblanc, Thermochromism in Commercial Products, J. of Chemical Education. 76.9 (1999) 1201.
[21] Windmiller, J. R., and J. Wang, Wearable Electrochemical Sensors and Biosensors: A Review. Electroanalysis 25.1 (2013) 29-46.
[22] Zhu, C., A. Wu, Studies on the synthesis and thermochromic properties of crystal violet lactone and its reversible thermochromic complexes, Thermochimica Acta. 425.1-2 (2005) 7-12.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Although the present invention has been described for the purpose of illustration, it is understood that such detail is solely for that purpose and variations can be made by those skilled in the art without departing from the spirit and scope of the invention which is defined by the following claims.

What is claimed is:

1. A process for making a thermochromic garment, said process comprising the steps of:
providing at least one thermochromic pigment-based coating solution comprising a unique thermochromic pigment having a desired activation temperature and activation color, a binding agent, and a thickener;
applying the at least one thermochromic pigment-based coating solution to a target surface of a fabric at a uniform thickness to yield a coated fabric having at least one thermochromic zone comprising the unique thermochromic pigment; and
curing the coated fabric using a heat treatment protocol effective to promote cross-linking, thereby yielding a thermochromic garment having at least one thermochromic zone.

2. The process according to claim 1, wherein said at least one thermochromic pigment-based coating solution comprises 4-10% of the unique thermochromic pigment, 18-20% of the binding agent, 10% of the thickener, and 70% of water.

3. The process according to claim 2, wherein said at least one thermochromic pigment-based coating solution is prepared by first providing the water and then mixing, in succession and while stiring vigorously, the binding agent, the unique thermochromic pigment, and then the thickener.

4. The process according to claim 1, wherein said unique thermochromic pigment comprises a leuco dye.

5. The process according to claim 1, wherein said unique thermochromic pigment has an activation color selected from the group consisting of blue, black, red, magenta, yellow, purple, violet, orange, white, and shades thereof.

6. The process according to claim 1, wherein said unique thermochromic pigment has an activation temperature selected from the group consisting of 31° C., 32° C., 33° C., 34° C., 35° C., 36° C., 37° C., 38° C., 39° C., and 40° C.

7. The process according to claim 1, wherein said unique thermochromic pigment has an activation color and an activation temperature corresponding to any combination of activation color and activation temperature, wherein said activation color is selected from the group consisting of blue, black, red, magenta, yellow, purple, violet, orange, white, and shades thereof, and wherein said activation temperature selected from the group consisting of 31° C., 32° C., 33° C., 34° C., 35° C., 36° C., 37° C., 38° C., 39° C., and 40° C.

8. The process according to claim 1, wherein said unique thermochromic pigment is reversible in that its activation color will revert to a non-activated color state when its activation temperature is not maintained.

9. The process according to claim 1, wherein the uniform thickness of the applied coating solution is a range selected from the group consisting of between about 0.1 millimeters (mm) and about 1.0 mm, between about 0.1 mm and about 0.8 mm, between about 0.1 mm and about 0.7 mm, between about 0.1 mm and about 0.6 mm, between about 0.1 mm and about 0.5 mm, between about 0.1 mm and about 0.4 mm, and between about 0.1 mm and about 0.3 mm.

10. The process according to claim 1 further comprising:
bleaching the fabric prior to applying the at least one thermochromic pigment-based coating solution to the target surface of the fabric, wherein said fabric comprises natural fibers.

11. The process according to claim 1 further comprising:
scouring the fabric prior to applying the at least one thermochromic pigment-based coating solution to the target surface of the fabric.

12. The process according to claim 11, wherein said scouring comprises submerging the fabric in a scouring solution comprising water, sodium hydroxide (NaOH), Invadine, and Invadex.

13. The process according to claim 11 further comprising:
bleaching the scoured fabric prior to applying the at least one thermochromic pigment-based coating solution to the target surface of the fabric, wherein said fabric comprises natural fibers.

14. The process according to claim 1, wherein said fabric comprises natural fibers, synthetic fibers, or a combination thereof.

15. The process according to claim 14, wherein the fabric comprising natural fibers is cotton.

16. The process according to claim 15, wherein the fabric comprising synthetic fibers is selected from the group consisting of spandex, nylon, polyester, and combinations thereof.

17. The process according to claim 1, wherein said heat treatment protocol of the curing step comprises drying the coated fabric at a temperature of about 100° C. and then further incubating the coated fabric at a temperature of about 160° C.

18. The process according to claim 1, wherein during the applying step the fabric is subtly stretched to facilitate adherence of the thermochromic pigment to the fabric.

19. The process according to claim 1, wherein a plurality of different thermochromic pigment-based coating solutions is provided for applying to the fabric,
wherein each thermochromic pigment-based coating solution comprises its own unique thermochromic pigment, and
wherein each thermochromic pigment-based coating solution is applied to a different target surface of the same fabric or to its own designated piece of fabric for assembly into the thermochromic garment,
thereby yielding a thermochromic garment comprising a plurality of different thermochromic zones.

20. The process according to claim 19, wherein the thermochromic garment further comprises non-thermochromic zones, said non-thermochromic zones not comprising a thermochromic pigment.

21. The process according to claim 21, wherein the non-thermochromic zone comprises a black colored or dark colored fabric and the thermochromic zones comprise white or substantially white fabric when the thermochromic pigments are not activated.

22. The process according to claim 20, wherein a plurality of different thermochromic zones can border or substantially border one another in order to provide a multi-part zone that exhibits a range of temperature activation.

23. The process according to claim 20, wherein a plurality of unique thermochromic pigments can be applied to the same target surface of the fabric so as to provide a thermochromic zone that exhibits different pigment colors at different activation temperatures.

24. The process according to claim 20, wherein the plurality of different thermochromic zones are located at regions corresponding to portions of a human selected from the group consisting of scalp, face, neck, upper back, lower back, shoulder, upper arm, tricep, inner arm, bicep, forearm, wrist, hand, waist, rear, groin, hip, upper thigh, lower thigh, inner thigh, calf, shin, achilles, foot, and portions thereof.

25. The process according to claim 1, wherein the at least one thermochromic zone is durable in that the corresponding unique thermochromic pigment contained in the thermochromic zone remains active after abrasion testing up to about 10,000 cycles and maintains color fastness according to benchmark apparel testing standards.

26. The process according to claim 1, wherein the process comprises integrating the thermochromic pigment to or into a fiber or textile precursor of the fabric prior to producing the thermochromic garment.

27. The process according to claim 26, wherein the fiber or textile is any fiber or textile used to make any garment suitable for wearable use by a human.

28. The process according to claim 1, wherein the thermochromic garment is made of any fabric, fiber, or textile suitable for use as a wearable garment for a human, including, but not limited to, fabrics, fibers, or textiles used for conventional garments.

29. The process according to claim 1, wherein the thermochromic garment is a type of garment selected from the group consisting of athletic wear, active wear, sportswear, casual wear, medical clothing, functional wear, underwear, and the like.

30. The process according to claim 1, wherein the thermochromic garment is in the form of garment selected from the group consisting of a shirt, shorts, pants, a sock, a glove, a headband, headwear (e.g., skull cap, hat, etc.), neckwear, a wrist band, an undergarment (e.g., upper body or lower body undergarments), compression wear (e.g., compression shorts, compression pants, compression stockings, compression torso wear, etc.), a sleeve (e.g., arm sleeve, leg sleeve, etc.), a wrap (e.g., ankle wrap, knee wrap, neck wrap, foot wrap, hand wrap, arm wrap, wrist wrap, leg wrap, etc.), and the like.

31. A thermochromic garment produced by the process according to any one of claims 1-30.

32. A method for monitoring changes in temperature of a human subject or of a portion of the human subject, said method comprising:
providing a thermochromic garment according to claim 31 to a human subject; and
monitoring changes in color of the thermochromic zones of the thermochromic garment while the human subject wears said thermochromic garment, wherein a change in the color of a thermochromic zone indicates a corresponding change in the temperature of the human subject or in a portion of the human subject.

33. The method according to claim 32, wherein the method relates to monitoring or treating various conditions or aspects of the human subject selected from the group consisting of physical exhaustion, diabetes, arthritis, general ulcers (e.g., skin ulcers), pressure ulcers, and changes in temperature of portions of the human subject.

34. A thermochromic garment comprising:
a fabric having a plurality of thermochromic zones and at least one non-thermochromic zone,
wherein each thermochromic zone comprises a thermochromic pigment having a desired activation temperature and a desired activation color, and
wherein the at least one non-thermochromic zone does not comprise a thermochromic pigment.

35. The thermochromic garment according to claim 34, wherein said thermochromic pigment comprises a leuco dye.

36. The thermochromic garment according to claim 34, wherein said thermochromic pigment has an activation color selected from the group consisting of blue, black, red, magenta, yellow, purple, violet, orange, white, and shades thereof.

37. The thermochromic garment according to claim 34, wherein said thermochromic pigment has an activation temperature selected from the group consisting of 31° C., 32° C., 33° C., 34° C., 35° C., 36° C., 37° C., 38° C., 39° C., and 40° C.

38. The thermochromic garment according to claim 34, wherein said thermochromic pigment has an activation color and an activation temperature corresponding to any combination of activation color and activation temperature, wherein said activation color is selected from the group consisting of blue, black, red, magenta, yellow, purple, violet, orange, white, and shades thereof, and wherein said activation temperature selected from the group consisting of 31° C., 32° C., 33° C., 34° C., 35° C., 36° C., 37° C., 38° C., 39° C., and 40° C.

39. The thermochromic garment according to claim 34, wherein said thermochromic pigment is reversible in that its activation color will revert to a non-activated color state when its activation temperature is not maintained.

40. The thermochromic garment according to claim 34, wherein the thermochromic pigment is on a surface of the fabric at a uniform thickness in a range selected from the group consisting of between about 0.1 millimeters (mm) and about 1.0 mm, between about 0.1 mm and about 0.8 mm, between about 0.1 mm and about 0.7 mm, between about 0.1 mm and about 0.6 mm, between about 0.1 mm and about 0.5 mm, between about 0.1 mm and about 0.4 mm, and between about 0.1 mm and about 0.3 mm.

41. The thermochromic garment according to claim 34, wherein said fabric comprises natural fibers, synthetic fibers, or a combination thereof.

42. The thermochromic garment according to claim 41, wherein the fabric comprising natural fibers is cotton.

43. The thermochromic garment according to claim 42, wherein the fabric comprising synthetic fibers is selected from the group consisting of spandex, nylon, polyester, and combinations thereof.

44. The thermochromic garment according to claim 43, wherein the thermochromic garment further comprises non-thermochromic zones, said non-thermochromic zones not comprising a thermochromic pigment.

45. The thermochromic garment according to claim 44, wherein the non-thermochromic zone comprises a black colored or dark colored fabric and the thermochromic zones comprise white or substantially white fabric when the thermochromic pigments are not activated.

46. The thermochromic garment according to claim 44, wherein a plurality of different thermochromic zones can border or substantially border one another in order to provide a multi-part zone that exhibits a range of temperature activation.

47. The thermochromic garment according to claim 44, wherein a plurality of unique thermochromic pigments is contained on a target surface of the fabric so as to provide a thermochromic zone that exhibits different pigment colors at different activation temperatures.

48. The thermochromic garment according to claim 44, wherein the plurality of different thermochromic zones are located at regions corresponding to portions of a human selected from the group consisting of scalp, face, neck, upper back, lower back, shoulder, upper arm, tricep, inner arm, bicep, forearm, wrist, hand, waist, rear, groin, hip, upper thigh, lower thigh, inner thigh, calf, shin, achilles, foot, and portions thereof.

49. The thermochromic garment according to claim 34, wherein the at least one thermochromic zone is durable in that the corresponding unique thermochromic pigment contained in the thermochromic zone remains active after abrasion testing up to about 10,000 cycles and maintains color fastness according to benchmark apparel testing standards.

50. The thermochromic garment according to claim 34, wherein the thermochromic garment is made of any fabric, fiber, or textile suitable for use as a wearable garment for a human, including, but not limited to, fabrics, fibers, or textiles used for conventional garments.

51. The thermochromic garment according to claim 34, wherein the thermochromic garment is a type of garment selected from the group consisting of athletic wear, active wear, sportswear, casual wear, medical clothing, functional wear, underwear, and the like.

52. The thermochromic garment according to claim 34, wherein the thermochromic garment is in the form of garment selected from the group consisting of a shirt, shorts, pants, a sock, a glove, a headband, headwear (e.g., skull cap, hat, etc.), neckwear, a wrist band, an undergarment (e.g., upper body or lower body undergarments), compression wear (e.g., compression shorts, compression pants, compression stockings, compression torso wear, etc.), a sleeve (e.g., arm sleeve, leg sleeve, etc.), a wrap (e.g., ankle wrap, knee wrap, neck wrap, foot wrap, hand wrap, arm wrap, wrist wrap, leg wrap, etc.), and the like.

53. A method for monitoring changes in temperature of a human subject or of a portion of the human subject, said method comprising:
providing a thermochromic garment according to any one of claims 34-52 to a human subject; and
monitoring changes in color of the thermochromic zones of the thermochromic garment while the human subject wears said thermochromic garment, wherein a change in the color of a thermochromic zone indicates a corresponding change in the temperature of the human subject or in a portion of the human subject.

54. The method according to claim 53, wherein the method relates to monitoring or treating various conditions or aspects of the human subject selected from the group consisting of physical exhaustion, diabetes, arthritis, general ulcers (e.g., skin ulcers), pressure ulcers, and changes in temperature of portions of the human subject.
